(19) United States
(12) Patent Application Publication
(10) Pub. No.: US 2025/0255477 A1
(43) Pub. Date: Aug. 14, 2025

(54) BIO-MEDICAL IMAGING DEVICES, SYSTEMS AND METHODS OF USE

(71) Applicant: PULSEMEDICA CORP., Edmonton (CA)

(72) Inventors: Nir Katchinskiy, Edmonton, Alberta (CA); Christopher Ceroici, Edmonton, Alberta (CA); Geoffroy Rivet-Sabourin, Stoneham-et-Tewkesbury, Quebec (CA)

(21) Appl. No.: 18/856,447

(22) PCT Filed: Apr. 14, 2022
(86) PCT No.: PCT/CA2022/050582

§ 371 Date: Oct. 11, 2024

(51) Int. Cl. A61B 3/00; A61B 3/024; A61B 3/12; A61F 9/008

(52) U.S. Cl. CPC A61B 3/0075; A61B 3/024; A61B 3/1208; A61B 3/1225; A61F 9/00821; A61F 2009/00846; A61F 2009/00863; A61F 2009/00874; A61F 2009/00891

(57) ABSTRACT

A modular ophthalmology device comprising: a first optical device having a light source and an imaging sensor, a first input coupling having an optical interface connection and a physical interface connection for aligning and securing an input optical module, a first optical output, an optical pathway and an output coupling securing a third optical device.

### TECHNICAL FIELD

The current disclosure relates to bio-medical imaging devices and systems and in particular to modular bio-medical imaging devices and systems.

### BACKGROUND

Imaging of organs, such as the eye, is important for identifying conditions of the organ. Various imaging techniques may be used for capturing images of the interior compartments of the eye. For example, scanning laser ophthalmoscopy (SLO) imaging may provide 2-dimensional image of a portion of the eye, such as the retina, vitreous, trabecular meshwork or of the cornea. Optical coherence tomography (OCT) imaging may provide 3-dimensional and/or cross-section images of a portion of the retina, vitreous, trabecular meshwork or of the cornea. Other imaging techniques may be used for capturing an image of at least a portion of the fundus of the eye.

Imaging of the eye may be used for identifying eye conditions requiring treatment. Treatment of eye conditions may be performed using lasers, with the specific targeting location of the laser beam or pulse determined from the captured images.

The imaging devices and the treatment laser may be incorporated into a single device. An additional, new and/or improved bio-medical imaging device capable of imaging and/or treating one or more conditions is desirable.

### SUMMARY

In accordance with the present disclosure there is provided an ophthalmology device comprising: a first optical device comprising a first light source and first imaging sensor for capable of generating image data using the first light source; a first input coupling comprising: a physical interface connection for aligning and securing an input optical module; and an optical interface connection; a first optical output; an optical pathway coupling the first optical device and the optical interface connection of the first input coupling to the first optical output; and an output coupling providing a physical interface connection for aligning and securing a third optical device.

In a further embodiment of the device, the device further comprises a controller for controlling operation of the device and providing one or more of: Calibration functionality for enrolling connected input modules and output modules for use with the device; image registration functionality; and retina tracking functionality.

In a further embodiment of the device, the first input coupling further comprises an electrical interface connection.

In a further embodiment of the device, the electrical interface connection provides a communication channel to the controller of the device.

In a further embodiment of the device, the device further comprises: a first tunable lens for the first optical device within the optical pathway; and a second tunable lens for the first input coupling within the optical pathway.

In a further embodiment of the device, the first tunable lens and second tunable lens are controlled by the controller in order to co-align light from the first optical device and the optical interface connection of the first input coupling.

In a further embodiment of the device, the device further comprises a second input coupling comprising: a physical interface connection for aligning and securing a fourth optical device; and an optical interface connection.

In a further embodiment of the device, the second input coupling further comprising an electrical interface connection.

In a further embodiment of the device, the output coupling further comprises an electrical interface connection.

In a further embodiment of the device, the optical pathway comprises one or more of: an optical splitter/combiner; an optical circulator; a filter; a galvanometer; a tunable lens; a lens; and a grating.

In accordance with the present disclosure there is further provided an ophthalmology system comprising: a base ophthalmology device according to any one of claims 1 to 10; and the input optical module secured to first input coupling.

In a further embodiment of the system, the input optical module comprises at least one of: an imaging device including a light source and sensor, optically coupled to the optical interface connection of the first input coupling; and a therapeutic laser.

In a further embodiment of the system, the input optical module further comprises one or more of: an optical device; a pilot laser; a graphical processing unit; and an additional input coupling.

In a further embodiment of the system, the input optical module provides one or more of: a 2D imaging system; a scanning laser ophthalmoscopy (SLO) imaging system; an optical coherence tomography (OCT) imaging system; and a therapeutic laser treatment system.

In a further embodiment of the system, the system further comprises an additional input optical module secured to the base ophthalmology device.

In a further embodiment of the system, the system further comprises an output optical module secured to the base ophthalmology device.

In a further embodiment of the system, the output optical module comprises one or more of: an optical device; a graphical processing unit; an additional input coupling; an additional output coupling; and a sensor.

In accordance with the present disclosure there is further provided a kit for an ophthalmology device comprising: a first optical module comprising: one or more optical components for providing ophthalmology functionality to the ophthalmology device; a physical interface connection for mechanically securing the first optical module to the ophthalmology device; an optical interface connection for optically coupling at least a portion of the one or more optical components to the ophthalmology device; and a module identifier for uniquely identifying the first optical module; and authorization for use of one or more software components by the ophthalmology device to provide the ophthalmology functionality using the first optical module identified by the module identifier.

In a further embodiment of the kit, the first optical module comprises one of an input module and an output module.

In a further embodiment of the kit, the first optical module comprises an input module, and the kit further comprises an output module.

In a further embodiment of the kit, the kit comprises at least one input module and output module suitable for the performing one or more of: central photocoagulation; peripheral photocoagulation; vitreous treatment with femtosecond laser; vitreous treatment with YAG laser; and glaucoma treatment with femtosecond laser.

In a further embodiment of the kit, the software component comprises functionality for performing automatic treatment planning using machine learning.

In accordance with the present disclosure there is further provided a method of operating a ophthalmology device comprising an internal imaging system and one or more input modules connected to the ophthalmology device, the method comprising: identifying the one or more input modules connected to the device; for each connected input module, determining if the connected input module comprises an optical component requiring alignment with the internal imaging system or other identified input modules; and for each input module requiring alignment, controlling a tunable lens of the device within an optical pathway that is optically coupled with the respective input module requiring alignment in order to align the respective input module.

In a further embodiment of the method, the alignment of the input modules aligns light paths of each of the input modules with a light path of the internal imaging system.

In a further embodiment of the method, the alignment of the input modules aligns a depth of focus to a common depth of focus of other components.

In a further embodiment of the method, the alignment of the input modules aligns a depth of focus to a different depth of focus from other components.

In a further embodiment of the method, the alignment uses image registration techniques to determine adjustments needed.

In a further embodiment of the method, the method further comprises using the image registration techniques to co-register images captured by the ophthalmology device.

In a further embodiment of the method, one or more output modules are connected to the ophthalmology device, the method further comprise: identifying the one or more output modules connected to the device; for each connected output module, determining if the connected output module comprises an optical component requiring alignment with the internal imaging system or other identified input and output modules; and for each output module requiring alignment, controlling a tunable lens of the device within an optical pathway that is optically coupled with the respective output module requiring alignment in order to align the respective output module

In a further embodiment of the method, the method further comprises: determining requirements and/or limitations of the connected input modules and output modules; determining one or more possible operating modes based on at least one of the requirements and limitations of the connected input modules and output modules; and enabling at least one of the possible operating modes.

### BRIEF DESCRIPTION OF THE DRAWINGS

Further features and advantages of the present disclosure will become apparent from the following detailed description, taken in combination with the appended drawings, in which:

FIG. 1 depicts components of an ophthalmology system;

FIG. 2 depicts components of a further ophthalmology system;

FIG. 3 depicts components of an ophthalmology device;

FIGS. 4A-4I depict components of various input modules for use with an ophthalmology device;

FIGS. 5A-5O depict components of various output modules for use with an ophthalmology device;

FIG. 6 depicts components of a further ophthalmology system;

FIG. 7 depicts an illustrative database for storing module registration data;

FIG. 8 depicts a method for using an ophthalmology system;

FIG. 9 depicts a further method for using an ophthalmology system; and

FIG. 10 depicts a method for calibrating an ophthalmology system.

### DETAILED DESCRIPTION

Bio-medical imaging systems and devices may be used to provide various imaging, as well as possibly treatment, options. The medical imaging systems and devices may be provided as a modular system. The medical imaging systems and devices are described further herein with particular reference to ophthalmic devices used for imaging and possibly treating an eye. Ophthalmic devices are designed to provide certain functionality for examining a patient's eye as well as possibly examining and/or treating an eye condition of the patient. These devices may include for example ophthalmoscopes for examining a fundus of the patient's eye, a tonometer for measuring pressure in the patient's eye, a visual field analyzer for testing a patient's field of vision, and laser treatment devices which may be used to photocoagulate, ablate, cut or otherwise treat ocular tissue. While these devices are useful for ophthalmologists, they can be expensive. Additionally, the numerous devices that may be needed can require a relatively large footprint and may require a patient to move from one device to another which can increase the time required to diagnose and/or treat a patient. As described further herein, an ophthalmology system may be provided that can be used for multiple purposes including the diagnosis and treatment of various eye conditions. The system comprises a base device that can provide an imager for performing imaging functionality of the patient's eye. In addition to the imager of the base device, the base device also includes a physical input coupler that allows additional optical input devices to be physically and optically connected to the base device. Various different optical input devices may be provided for providing different functionality including for example additional imaging and/or treatment devices. In addition to the physical input coupler, the base device also provides an output physical coupler that allows optical output devices to be physically and optically coupled to the base device. The output optical devices may provide various functionality such as possibly providing additionally focusing or defocusing optics. The base device along with the possible different input and output optical modules can provide ophthalmologist with a single device that can provide a wide range of functionality for examining, diagnosing and/or treating different eye conditions using the single device. The ophthalmology system described herein provides a cost effective device that can be used in place of possible multiple devices.

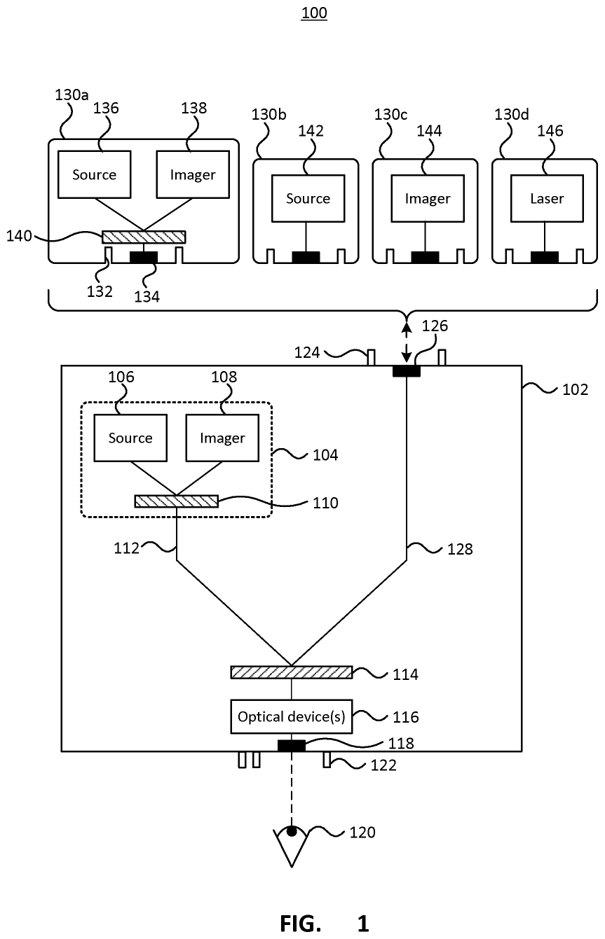

FIG. 1 depicts components of an ophthalmology system. The ophthalmology system 100 comprises a base device 102 that can provide base functionality. The base functionality 104 is depicted in FIG. 1 as an imaging device that includes a light source 106 as well as an imager or image sensor 108 that can detect the light from the source and generate image data. The light source may be a visible and/or non-visible light source. Additionally, the light source may be a coherent and/or non-coherent light source. A beam splitter device 110 is provided that allows the light from the light source 106 to pass to an optical pathway 112 of the base device and provides light returning from the optical pathway 112 to the imager 108. The beam splitting device may be provided in various ways depending on the light source and imager. For example, the beam splitter could be provided as a two-way mirror that allows light from the source to pass through but reflects returning light to the imager. Alternative devices may include polarizing and non-polarizing beam splitters, optical circulators or other arrangements.

Regardless of the specific implementation of the source 106, imager 108 and beam splitter 110 of the base imaging functionality 104 are optically coupled to an optical path 112 of the base device. The optical path 112 may couple to another beam combiner/splitter device 114, which may be for example a dichroic mirror, and one or more optical devices 116. The optical devices 116 may comprise one or more lenses that can adjust the focus of the light from the source passing through an optical output port 118 of the base device 102 onto the patient's eye, or other target, 120. The optical device may comprise other optical devices such as filters, adjustable mirrors, shutters or apertures, adaptive optics, etc. The light from the source may return from the patient's eye though the output port 118 and back to the optical pathway 112 where it is provided to the imager 108 in order to generate image data of the eye or target.

The base device 102 further includes a physical output coupler 122 that can be used to align and lock an optical output module (not shown in FIG. 1). The physical output coupler 122 is depicted as three pins extending downward from the device; however it will be appreciated that the physical coupling device may be provided by various physical connectors that can physically locate and align an output module to the base device and lock the module in place. The base device 102 further comprises a physical input coupler 124 that includes a physical alignment component, depicted as a pair of pins, as well as an optical input port 126 that provides an optical coupling between an input optical module connected to the input coupler 124 to a second optical path 128 that is optically coupled to the second beam splitter 114 and so the optical device(s) 116 and optical output port 118. The output physical coupler 122 and the input physical coupler 124 are depicted as providing a different physical interface, with the output coupler using three pins and the input coupler using two pins, in order to prevent input modules and output modules from being incorrectly coupled to the base device. Although depicted as providing a different physical coupling interface, the output and input couplings may use the same physical interface.

The base device 102 provides imaging functionality 104 as well as a physical connector 124 for connecting one or more input modules to the base device. Various input modules 130a, 130b, 130c, 130d (referred to collectively as input modules 130) are depicted in FIG. 1. Each of the modules includes a respective physical interface 132 that engages with the physical interface 124 of the base device in order align and secure the input modules to the base device. The input modules 130 also comprise an optical interface 134 that is optically coupled to the optical input port 126 when the input module is physically coupled to the base device.

Each of the input modules 130 may be used to provide different functionality. For example, input module 130a is depicted as provide a light source 136 and imager sensor 138 and a beam splitter 140. The input module 130a may provide an alternative device compared to the imaging functionality 104 provided by the base device. An additional input module 130b is depicted as providing an additional light source 142, which could provide for example improved lighting, or lighting of a different bandwidth, for imaging by the image device. For use with such an input module, the beam splitter 114 should return, at least, a portion of the light from the source 142 to the imager 108. A further input module 130c is depicted as providing an imager 144, which may capture alternative, additional and/or improved image data using the light source 106 of the base device. In use with such an input module, the beam splitter 114 should return, at least, a portion of the light from the source 106 of the base device. A further input module 130d is depicted that provides a laser source 146 that can be used possibly as a light source for an imaging device or as a therapeutic laser for treatment of the patient's eye 122.

FIG. 2 depicts components of a further ophthalmology system. The system 200 is similar to that described above with reference to FIG. 1, and comprises a base device 202 that has input and output modules physically and optically coupled to it. The base device 202 provides imaging functionality 204 with an imaging source 206 and imaging sensor 208. The imaging functionality 204 may incorporate additional optics and/or components not depicted in FIG. 2. The imaging source and sensor may vary depending upon the imaging functionality provided. For example, the imaging functionality may provide a digital ophthalmoscope using a white light source and a 2D image sensor, such as a CMOS sensor along with appropriate optics. Alternatively, the imaging functionality may provide SLO (Scanning Laser Ophthalmoscopy) functionality that uses a laser source with scanning optics such as galvanometers, resonant scanners, spinning mirrors for scanning the laser across the patient's eye to generate an image of the eye.

The base device 202 has an optical path 212 that provides an optical path between the imaging functionality 204 and a beam combiner/splitter device 214 that is optically coupled to one or more optical devices 216 such as focusing optics. The focusing optics may comprise one or more adjustable lenses. Additionally or alternatively, the optical devices may comprise scanning optics for controlling a direction of the light on the patient's eye. The base device 202 comprises an optical output port 218 that allows the optical devices to be optically coupled to a corresponding optical coupler of an output optical module 220 that is mechanically, electrically, and data transfer coupled to the base device by a physical interface connector 222. The physical connection may comprise cooperating features on both the base device and the output module 220.

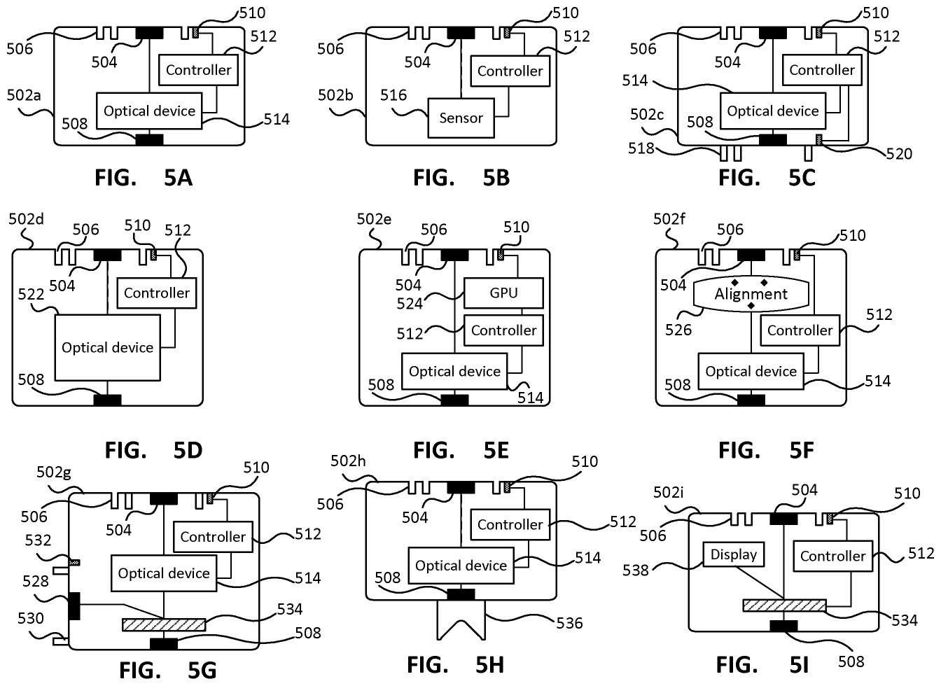

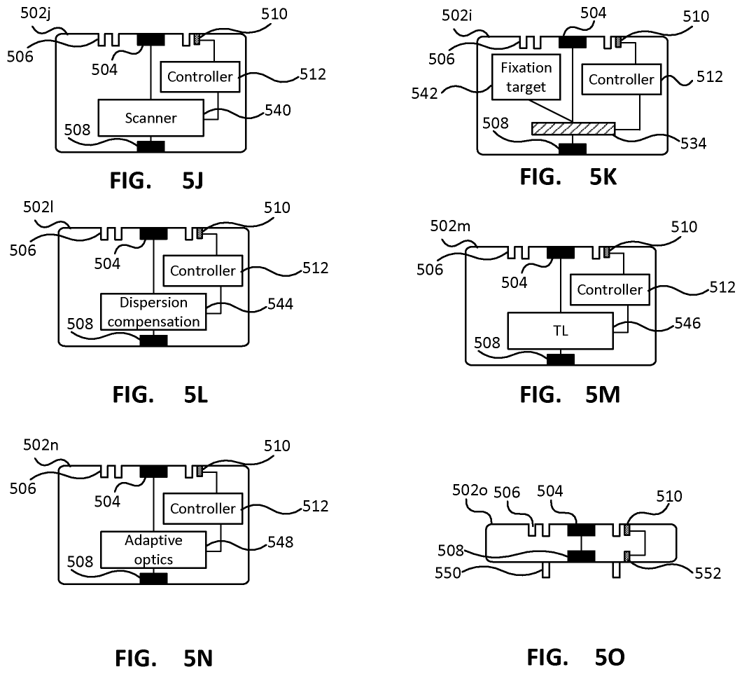

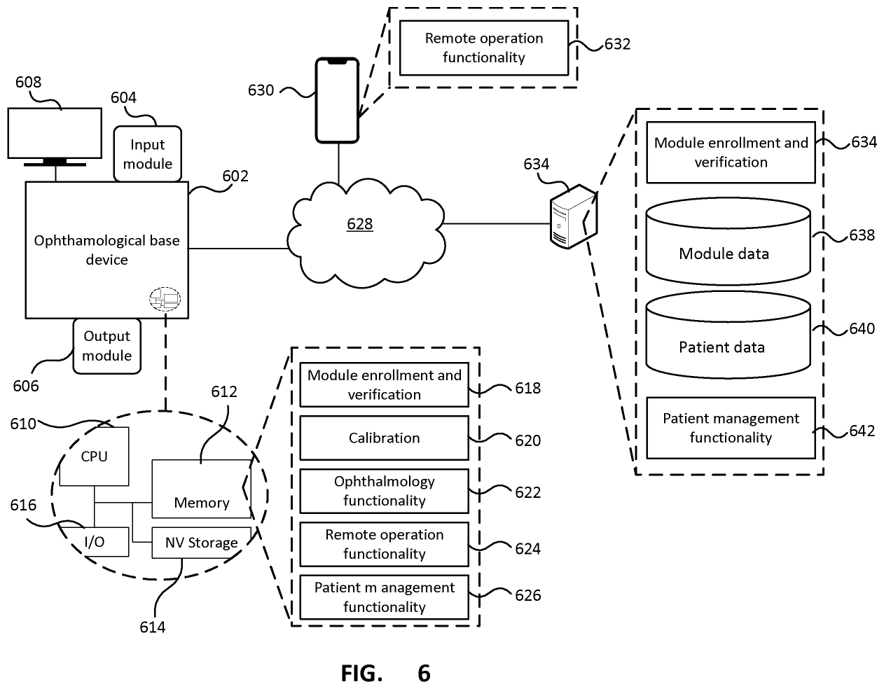

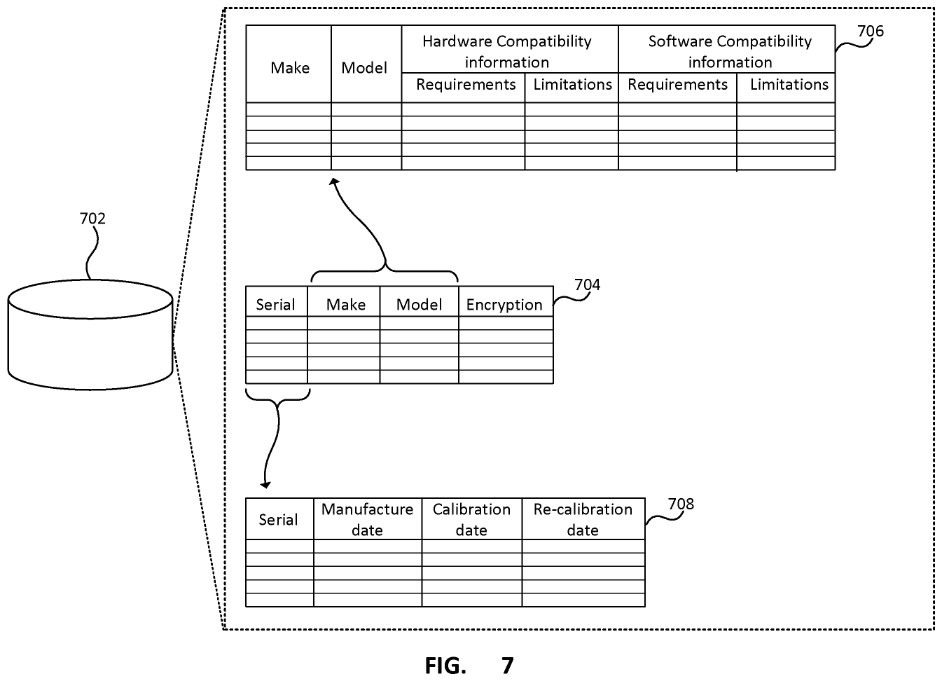

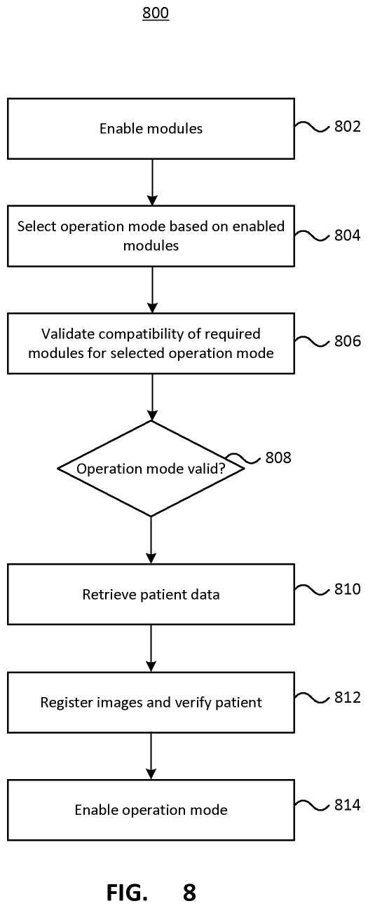

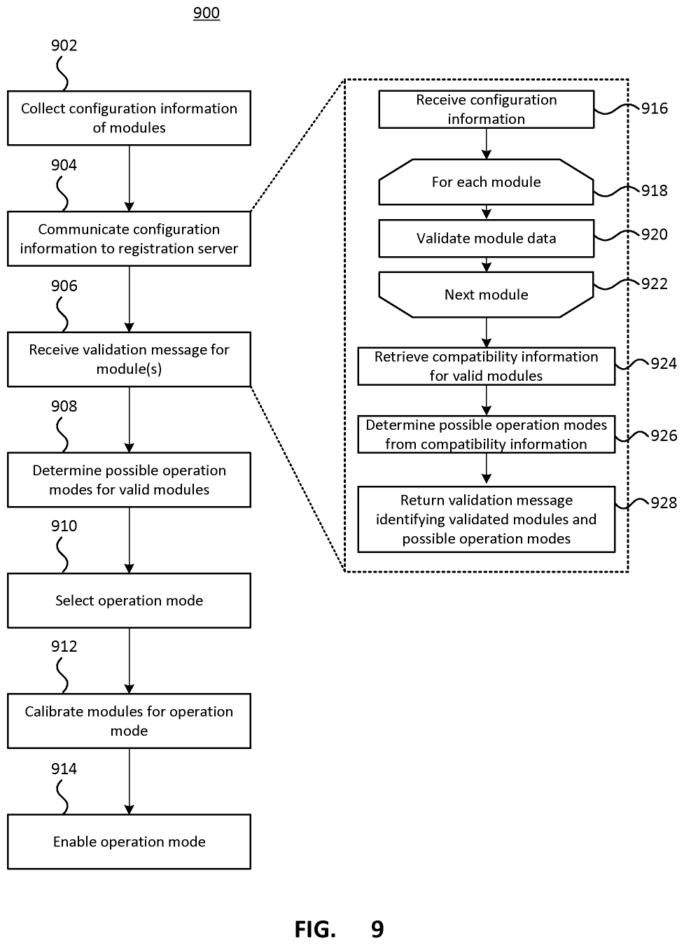

The output module 220 is depicted as providing optics 224 that comprise movable lenses 224a, 224b that can be used to adjust a focusing depth of the light source and imager through an output port 228 onto a patient's eye 230. Other output modules that comprise various different components for providing differing functionality may be provided. Example output modules are described further below with reference to FIGS. 5A-5O.

In addition to the output module 220, the ophthalmology system 200 comprises an input optical module 232 that is mechanically coupled to the base device 202 using a physical interface 234 between the module and the device. The physical interface 234 may comprise cooperating features on each of the input module 232 and base device 202 that align and lock the input module in place relative to the base device. As depicted, the input module 232 provides a further light source 236 and corresponding imaging sensor 238 along with a beam combiner/splitter or other optical devices 240 for directing the light from the source to an optical interface 242 between the input module 232 and the base device 202 and from the optical interface 242 to the sensor 238. The optical interface 242 couples the input module to a second optical path 244 of the base device 202 that in turn is optically coupled to the output module 220 through the beam combiner/splitter 214 and optical devices 216.

The input module may provide a different imaging modality from the imaging functionality 204 of the base device. For example, the base device may be provided with SLO imaging functionality while the input module 232 may provide OCT (Optical Coherence Tomography) imaging. It will be appreciated that not all of the imaging components for providing OCT imaging are depicted in FIG. 2.

The base device 202 may further include a controller 250 for controlling operation of the ophthalmology system 200. The controller may control operating of the imaging functionality of the base device as well as providing a user interface for operating the system 200. The user interface may be provided as a graphical user interface displayed on a display (not shown) of the base device or may comprise an interface provided on a remote device that may be in communication with the controller via a communication module such as a Wi-Fi® radio, NIC (Network Interface Card), serial communication interfaces or other communication devices such as Bluetooth radios. The controller 250 may be provided as one or more individual control devices such as FPGAs (Field Programmable Gate Arrays), ASICs (Application Specific Integrated Circuits), and/or Central Processing Units (CPUs) that are operating together to provide the functionality of the system 200.

The base devices 102, 202 described above provide internal imaging functionality as well as interfaces for coupling, both mechanically and optically, input and output modules to the base device. In addition to the physical and optical interfaces between the input/output modules and the base device, an electrical interface 246 for the output module and an electrical interface 248 for the input module. The electrical interfaces 246, 248 provide one or more electrical connections between the modules and base device. The electrical interfaces may couple the input/output modules to the controller 250 of the base device in order to provide electrical power to components of the input/output modules as well as provide a communication path. As depicted the input and output modules may each include one or more controllers 246, 248. The controllers of the modules may communicate with the controller of the base device in order to co-ordinate operation of the modules. Additionally, the controllers of the input and output modules may provide information useful for configuring the modules with the base device. For example, the controller 252 of the output module may communicate identifying information stored in the module to the controller of the base device 202. The identifying information may provide information to the base device on how the base device can control the output module as well as other information such as possibly other requirements and/or limitations of the output module.

As described above, the controller of an output or input module may communicate identifying information about the module. The identifying information may be provided in various ways such as using an RFID tag 256 on the module and an RFID tag reader 258 on the base device 202 that can communicate the module's identifying information stored in the RFID tag. In addition to providing identifying information to the base device, the base device may also provide identifying information of the base device to the modules to allow the modules to identify the base device and possibly adjust the operation of the input or output module to operate with the base device.

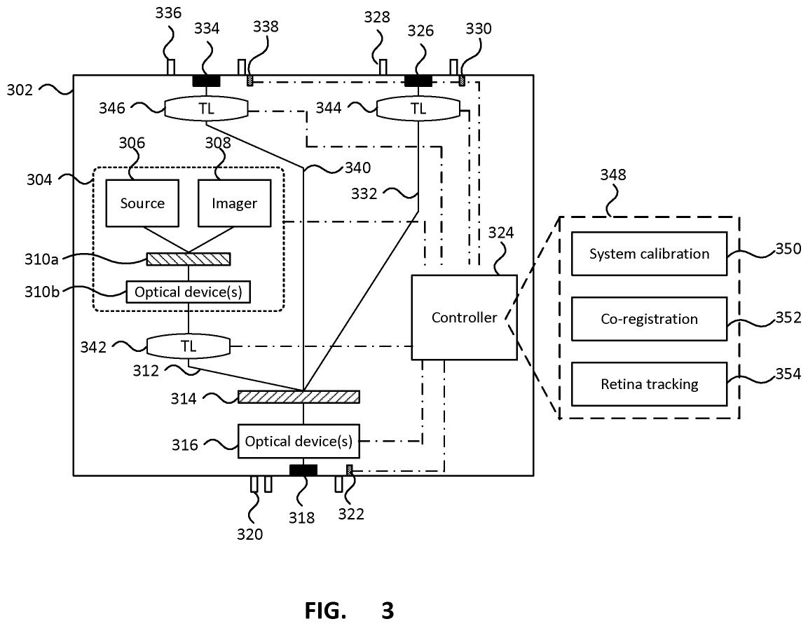

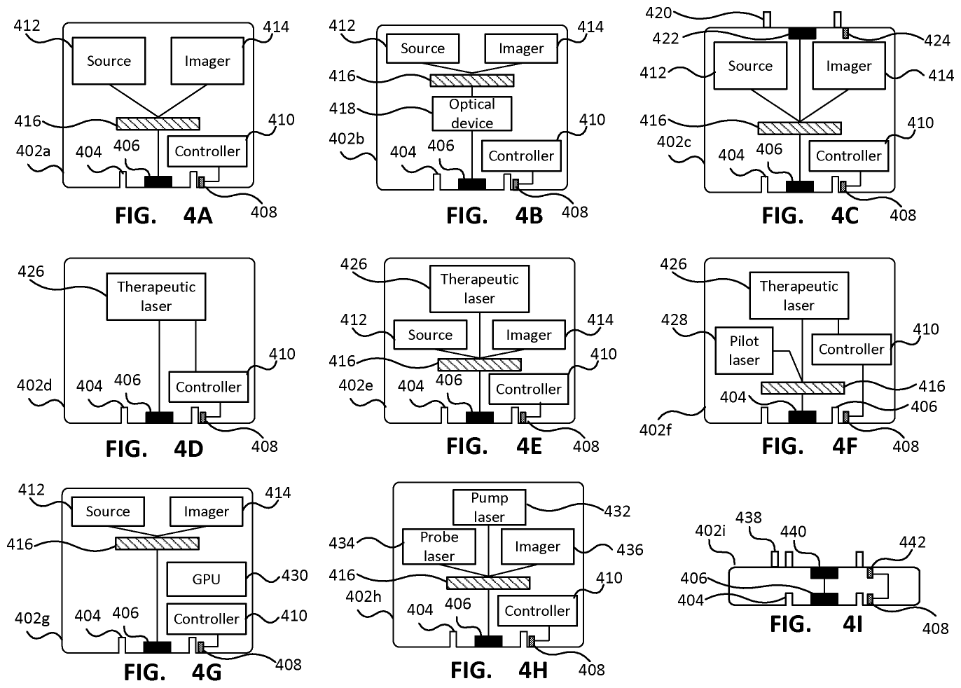

Using a base ophthalmology device as described above, it is possible to provide additional modules, whether input and/or output modules that can extend the functionality of the base device. Different input and output modules may be combined in different kits to provide various functionality. Illustrative input and output modules are described further below with reference to FIGS. 4A-4I and 5A-5O.

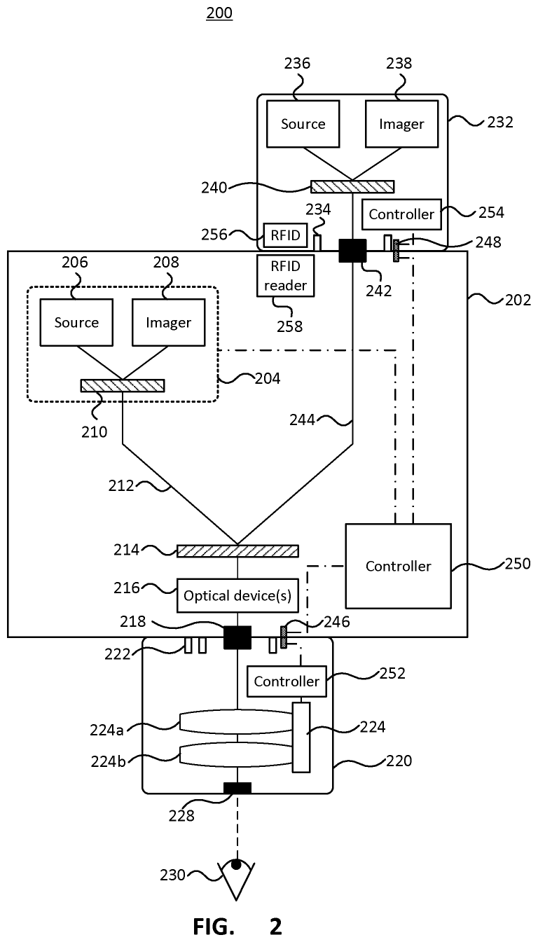

FIG. 3 depicts components of an ophthalmology device. The base device 302 is similar to the base devices 102, 202 described above. The base device 302 comprises imaging functionality 304 provided by, for example, a light source 306 and imaging sensor 308 coupled to a common optical path by a combiner/splitter device, or devices 310a. Additional optical devices 310b may be included as part of the internal imaging. The optical devices 310b may comprise, for example, focusing lenses and/or scanning/targeting optics, slits and/or apertures, and/or adaptive optics and/or dispersion compression/compensation optics, and/or spatial light modulators, and/or scanning optics such as spinning mirrors and/or galvanometers, and/or reference arms. Light for the imaging functionality 304 is coupled to an optical path 312 of the base device that passes through an optical combiner/splitter device 314 and one or more optical devices 316 such as lenses, filters, scanning optics, etc. The optical path terminates at an optical output port 318 that can either deliver the light to/from a patient's eye or other device such as a surgical lens or to/from an optical output module. A mechanical connection interface 320 is provided in order to align and secure the output module to the base device in an orientation that will couple the optical output port of the base device with an optical port of the output module. Similarly, an electrical interface connection 322 may be made between the output module and the base device when the output module is physically connected to the base device. The electrical connection may provide an electrical communication path between a controller of the output module and a controller 324 of the base device.

An input module can be similarly connected to the base device. The input connection may include an optical interface connection 326, a physical interface connection 328 and an electrical interface connection 330. The optical interface connection 326 can provide an optical path between the input module and a second optical path 332 of the base device connected to the combiner/splitter device 314. The base device may comprise additional connections for input modules which each may include an optical interface 334, physical interface connection 336 and electrical interface connection 338. The optical interface connection 334 may couple a connected module to a third optical path 340 and optical combiner/splitter 314 of the base device.

The base device is depicted as further comprises a plurality of tunable lens (TL) devices 342, 344, 346. Each tunable lens is arranged in an optical path from an input module or the imaging functionality. Although not depicted in FIG. 3 the optical devices 318 of the base device arranged at the output may include a tunable lens or provide other optical devices that function as a tunable lens. The tunable lenses can be individually controlled in order to adjust the focus of the light in the respective optical paths. The tunable lenses can be controlled in order to co-align devices. For example, if one of the input modules provides an imaging functionality in addition to the imaging functionality of the base device, the two tunable lenses can be controlled, for example by the controller 346, in order to co-align the two imaging devices so that positions within one of the imaging devices match positions within the other imaging device. Similar functionality may be provided to co-align another input module, which may provide for example a therapeutic laser, to one or more of the imaging devices. The co-alignment of the devices may be performed in various ways, including for example identifying common features across the imaging systems and controlling one or more of the tunable lenses to adjust the position of the common feature in the images captured by one or more of the imaging devices. Although described as a tunable lens, the same or similar functionality may be provided by other optical devices such as adaptive optics such as a spatial light modulator.

The importance of the co-alignment may depend upon the application of the device. For example, if the device is used for imaging a patient's eye for a general examination, the co-alignment of images captured by the different imaging devices, that may be either internal to the device or provided by connected modules, may not be of great importance as the captured images are intended to be reviewed individually by a physician and so the co-alignment of individual features is not of great importance. Alternatively, if the images are being analyzed in conjunction with each other possibly to diagnose an eye condition, it may be more important that the images be co-aligned, however in the case of diagnosis it may not be of critical importance to have the physical systems co-aligned as the images may be registered to each other by processing after being captured. If the system is being used for treatment of an eye condition, for example using a treatment laser input module connected to the base system, it may be of critical importance that the laser and imaging system used to determine targets for treatment are co-aligned so that targets identified in an image captured by one or more imaging devices are correctly targeted by the treatment laser. Accordingly, operation of the tunable lenses and the precision with which the connected optical devices are co-aligned may depend upon the current application.

Additionally, or alternatively, the tunable lenses 342, 344, 346 may be used to adjust a depth of focus of each optical device. Depending upon the particular arrangement of optical devices that are within the base device and connected to it, different applications may be provided and may require different depth of focus of the system. For example, if the internal imaging device of the base device is an SLO imaging device, and an input module comprises an OCT imager and treatment laser that passes through the scanning optics of the OCT imager and is co-aligned with the OCT source laser, it is possible to image and treat various eye conditions. For example, in the case of imaging and treating a retinal tear, both the SLO and OCT with the treatment laser should have the associated tunable lenses configured to provide both optical devices the same depth of focus, as well as being aligned with each other such that points in one imaging system can be directly mapped to points in the other imaging and treatment system. Such a co-alignment and depth of focus allows tears in the retina to be identified using the aligned SLO and OCT images and then treat the tear using the treatment laser. In contrast, the same SLO and OCT imaging and treatment system may be configured to treat a different condition such as floaters. In such a case, the tunable lens of the SLO may focus the depth of the imaging at the retina in order to capture images of the floaters, or their shadows on the retina. The SLO images may then be used to target the OCT imaging and treatment system, however the tunable lens of the OCT imaging and treatment system is used to adjust the depth of focus within the vitreous humor where the floaters are located. Once a floater is identified within the vitreous humor, the treatment laser, which is aligned with the OCT leaser and passes through the same scanning and focusing optics, may be operated to target and break up the floater. Different optical systems may be co-aligned such that their beams are co-linear with each other along the optical path. Although beam may be aligned with each other, they may be focused at different depths/lengths along the optical path.

The controller 324 may provide various functionality 348 including, for example system calibration functionality 350, image co-registration functionality 352 and retina tracking functionality 352. This functionality is only an example and other functionality may be provided. The calibration functionality 350 can determine which connected components comprise optical elements that require calibration, for example in order to align the optical elements. The calibration can comprise controlling a tunable lens that is within the optical pathway of the device in order to align the optical element. The alignment may be to a desired reference location, such as a center of a lens, or may be relative to other components. The calibration functionality adjusts components of the system in order to operate together in order to provide desired functionality. The calibration may include adjusting optical devices including tunable lenses in order to align different optical devices with each other, adjust depths of focus, adjust operating parameters of light sources, and sensors, etc. The calibration process may be performed at one or more times including for example, initially when setting up the system, when modifying the system by adding or removing modules, when starting or re-starting the system, when working with a new patient, and/or when desired or required by a user.

The co-registration functionality 352 may allow two or more images to be registered to each other. The registration process may identify common features in one of the images and matches them to corresponding features in other images. The registration may be used to provide a mapping that maps positions of common features in one of the images to the positions of the corresponding features in the other images. The mapping may be used to align the optical systems or for other processes. The co-registration may be applied to images of the same modalities or different modalities. Additionally, or alternatively, the co-registration may be applied to current images captured from imaging devices of the system, and/or previously captured by the system or using other imaging devices.

The retina tracking functionality 354 may be used to track movement of a patient's eye during use of the imaging system. The retina tracking may be used to adjust imaging parameters such as a target location and depth of focus. While the images are described as individual images, the images may be captured as a video stream of images. The retina tracking may be applied across images of a stream to track the patient's eye movement in time. The retina tracking may be applied to the whole image or frame and/or to sub frame strips of the image or frame.

FIGS. 4A-4I depict components of various input modules for use with an ophthalmology device. The input modules 402a-402g (referred to collectively as input modules 402) depicted in each of FIGS. 4A-4I each comprise a physical interface connection 404 that allows the input modules to be physically secured to the base device. The physical interface connection 404 provides one or more features that allow the input modules to be aligned and secured in position so that an optical port 406 of the input module is arranged with and optically coupled to the corresponding optical path of the base device. Alternatively, the optical connection may be established before physically connecting the input module to the base device using the physical interface connection. The input modules may also have an electrical interface connection 408 for electrically connecting the input modules to the base device. The input modules 402 may each include one or more controllers 410 for use in operating the input module with the base device, possibly along with other input modules and one or more output modules.

Turning to FIG. 4A, the input module 402a is depicted as providing an imaging device that includes an imaging light source 412, which could be for example a non-coherent light source such as a white light, a coherent light source such as a laser, which may be a continuous laser or pulsed laser operating at for example femtoseconds, picoseconds, or nanoseconds. The light source may provide light of one or more wavelengths within the visible and invisible spectrum. The input module 402a further includes an imaging device, such as a CMOS sensor, photodiode, avalanche photodiode, balanced photodetector, or other sensor that can capture light from the source 412 returning from impinging the patient's eye. A combiner/splitter device 416 directs light from the source 412 to the optical port 406 of the input module, and returning light from the optical port 406 to the imager sensor 414. The sensor 414 may communicate captured image data to the controller 410, which may in turn communicate the image data to the base device over the electrical interface 408.

Turning to FIG. 4B, the input module 402b is similar to the input module 402a however includes one or more additional optical devices 418, which may be arranged between the combiner/splitter 416 and optical port 406. The optical devices 418 may comprise, for example, focusing lenses and/or scanning/targeting optics, slits and/or apertures, and/or adaptive optics and/or dispersion compression/compensation optics, and/or spatial light modulators, and/or scanning optics such as spinning mirrors and/or galvanometers, and/or reference arms. The input module 402b may be used to provide various functionality such as SLO imaging, OCT imaging, hyperspectral imaging, Raman spectroscopy, slit lamp imaging, etc.

Turning to FIG. 4C, the input module 402c is similar to the input module 402a, however the input module includes an input module interface connection providing a physical interface 420 for connecting an additional input module to the input module 402c. The input module 402c further includes an optical port for coupling the additional input module to the combiner/splitter device 416 of the input module 402c.

Turning to FIG. 4D, the input module 402d comprises a therapeutic laser 426 that can be used in treating an eye condition. Although not depicted in FIG. 4D, the input module 402d may include one or more optical devices such as focusing lenses and/or scanning/targeting optics, and/or adaptive optics and/or dispersion compression/compensation optics, and/or spatial light modulators, and/or scanning optics such as spinning mirrors and/or galvanometers, and/or reference arms. Additionally or alternatively, the optics of the base device may be used in focusing and/or targeting or otherwise manipulating the therapeutic laser. Further, the input module 402d may comprise one or more safety devices to ensure that the therapeutic laser 426 is not able to fire unless it is determined safe to do so as well as to ensure that if there is a failure of one or more components, the input module fails in a safe mode in which the therapeutic laser cannot be fired.

Turning to FIG. 4E, the input module 402e provides a combination of the input module 402a and the input module 402e. The input module 402e may provide an imaging system and treatment system that can target and treat a condition in the eye. The input module 402e may include additional optical components such as focusing lenses and/or scanning/targeting optics, and/or adaptive optics and/or dispersion compression/compensation optics, and/or spatial light modulators, and/or scanning optics such as spinning mirrors and/or galvanometers, and/or reference arms. Further, additional optical devices may be provided for aligning the imaging device, or the source 412 and imager 414, with the therapeutic laser. The alignment ensures that the source 412 and therapeutic laser a co-linear along the optical path.

For example, if the imaging device is an OCT imaging device, the source 412 is a laser source, and when the therapeutic laser is fully aligned with the OCT source laser the two laser beams or pulses will be co-aligned so that the beams are co-linear with each other along the entire optical pathway they have in common. The alignment components may also be used to align the therapeutic laser with the optical port 404 of the input module. The alignment components may include both coarse alignment components and fine alignment components. The coarse alignment components may include for example positioning optics arranged in a Z-fold arrangement, a FIG. 4 arrangement or any other type of arrangement suitable for aligning the therapeutic beam. After passing through the positioning optics the therapeutic beam may pass through a beam splitter that directs a small portion of the beam to coarse alignment sensors and the other portion to the optical coupler 416 of the input module 402a. The light split for alignment is further split by a second beam splitter for directing the light into two separate paths, of differing lengths that terminate at respective sensors that can determine the incident location of the light in two orthogonal axis, such as the X and Y axis. The sensors may be for example CMOS sensors which provide a relatively large sensor area in order to be able to detect the incident location even if the beam is relatively poorly aligned. The fine alignment components may be similar to the coarse alignment although rather than using the CMOS sensors, the detection sensors use two quadrature photodiodes (QPD) that provide more precise location detection of incident lasers. As with the coarse alignment the path lengths to each QPD should differ to ensure the path of the beam is aligned along the path. That is, if the path lengths were the same, the sensors would only confirm that the path was aligned at the particular location, but the beams could be diverging or converging from the point.

Although the above has described alignment components with respect to the input module 402e, similar alignment components can be provided in other input modules, and/or the base device itself in order to align one or more components with each other. Depending upon the alignment precision required, the alignment components may be omitted entirely or one or more of the coarse and fine alignment components may be provided., only the coarse alignment

Turning to FIG. 4F, the input module 402f is similar to the input module 402e, however instead of providing an imaging light source 412 and image sensor 414, the input module 402e provides a pilot laser 428 along with the therapeutic laser. The pilot laser 428 may be a relatively low powered laser light source and may have similar/different wavelength that may be captured by another imaging sensor of, for example the base device or additional input modules or output modules. The pilot laser may be aligned with the therapeutic laser and used to verify that the correct location within a patient's eye will be targeted by the therapeutic laser prior to treatment with the therapeutic laser. The input module may include additional components not depicted such as optical devices including focusing lenses and/or scanning/targeting optics, and/or adaptive optics and/or dispersion compression/compensation optics, and/or spatial light modulators, and/or scanning optics such as spinning mirrors and/or galvanometers, and/or reference arms as well as alignment components.

Turning to FIG. 4G, the input module 402g is similar to the input module 402a however, the input module includes additional processing resources, depicted as a graphical processing unit (GPU) 430. The additional processing resources may be used directly by the input module in order to process images captured by the imager 412 prior to providing the processing results to the base device. Additionally or alternatively, the additional processing resources may be used directly by the base device, for example to process image data captured by components other than the components of input module 402g. In such embodiments, the electrical interface connection between the input module 402g and the base device may provide a high-speed and high-bandwidth communication interface.

Turning to FIG. 4H, the input module 402h is similar to input module 402F. The input module comprises a pump laser 432 that can be used to excite a target sample and a probe laser 434 and imager 436 for the probe laser that can image the excited target sample. The input module may include additional components not depicted such as optical devices including focusing lenses and/or scanning/targeting optics, and/or adaptive optics and/or dispersion compression/compensation optics, and/or spatial light modulators, and/or scanning optics such as spinning mirrors and/or galvanometers, and/or reference arms as well as alignment components.

Turning to FIG. 4I, the input module 402i does not include optical components, but instead is used as a physical adapter. In addition to the input connection 404, 406, 408, the input module provides another connection comprising a physical interface 438, optical interface 440 and electrical interface 442. The optical interfaces 406 440 are optically coupled together. The electrical interfaces 442 may be connected together. Although not depicted, the adapter may also include a controller. The controller may convert the signals between the electrical interfaces if necessary as well as provide information about the module to other connected modules or devices.

The above has described a plurality of different input module using the same physical, optical and electrical interface connections. It is possible for the base device to use one or more different physical, optical and/or electrical interface connections. For example, a first physical interface, optical interface and electrical interface may be provided for use with input modules providing relatively simple imaging functionality, while a separate physical interface, optical interface and electrical interface can be provided for input modules providing therapeutic lasers. As an example, one interface may provide a higher quality of connection, in terms of the precision of the alignment of the input modules, the quality of the optical connection and the bandwidth provided by the electrical connections.

The above input modules have been described individually. It is possible for one or more components described with reference to one input module to be combined with components from one or more other input module. Similarly, components described with regard to a single input module may be provided in separate modules.

FIGS. 5A-5O depict components of various output modules for use with an ophthalmology device. The output modules 502a-502h (referred to collectively as output modules 502) depicted in each of FIGS. 5A-5H each comprise a first optical port 504 for optically coupling the output modules to the base device along with a physical interface connection 506 that allows the output modules to be physically secured to the base device. The physical interface 506 is depicted as being physically different from the physical interface connection of the input modules so that input and output modules cannot be incorrectly attached to the base device. The physical interface connection 506 provides one or more features that allow the output modules to be aligned and secured in position so that the optical port 504 of the output module is arranged with and optically coupled to the corresponding optical path of the base device. Alternatively, the optical connection may be established before physically connecting the output module to the base device using the physical interface connection. The output modules 502a and 502c-502h each comprise an output optical port 508 that provides an optical port for the output of light, or input of returning light. The output modules 502 may also have an electrical interface connection 510 for electrically connecting the output modules to the base device. The output modules 502 may each include one or more controllers 512 for use in operating the output module with the base device, possibly along with other output modules and one or more input modules.

Turning to FIG. 5A an output module 502a is depicted provides optical devices 514 in the optical pathway between the optical port 504 and optical port 508. The optical devices 514 may include filters, apertures, lenses, mirrors, focusing lenses, scanning/targeting optics, adaptive optics, dispersion compression/compensation optics, spatial light modulators, scanning optics such as spinning mirrors and/or galvanometers, reference arms, alignment components, etc. The optical devices allow the light passing through, or reflecting/refracting off, the optical device to be manipulated. The optical devices 514 may be used to, for example filter light, focus the light, and/or scan or target the light. The optical devices 514 may be passive optical devices or may be active optical devices whose optical characteristics can be controlled, for example by the controller 512. For example, the output module 502a could provide a variable focus depth to allow imaging of different portions of the eye, such as the retina as well as the lens.

Turning to FIG. 5B, an output module 502b is depicted that provides an optical sensor 516. Although depicted as a single optical sensor, the optical sensor 516 may be provided as a plurality of different optical sensors and different optical devices such as lenses, beam splitters/combiners, filters, etc. The output module 502b may provide one or more sensors and may provide various alignment and/or calibration and/or configuration functionality. For example, the output module 502b may provide various alignment sensors to ensure different imaging components of the ophthalmology device are aligned. The sensor readings may be provided to the controller of the base device and used to adjust the other components to ensure they are properly aligned. Additionally or alternatively, the output component may have one or more calibrated sensors that may be used to calibrate, or verify the calibration of other components. For example, the sensor may provide a power sensor that can determine a power of a laser and either used to configure the laser device to provide certain power levels, or to verify that the laser device is providing particular power levels. Additional or alternative sensors could be provided to determine spectrums of light. The data provided sensor(s) 516 may be used to align components, verify components are working within operational thresholds, adjust components to operate within operational thresholds etc.

Turning to FIG. 5C, output module 502c is similar to output module 502a however includes an additional physical connection 518 and electrical interface connection for connecting to another output module. The output module 502c may provide the same functionality as output module 502a while allowing additional output modules to be coupled to the device. For example, the output module 502c may allow a calibration output module 502b to be coupled to the output module 502c. The output module may include one or more components whose quality may degrade over time and by attaching the calibration module 502b to the output module 502c the operating parameters of the output module 502c can be verified to still be within the allowable thresholds and as such may still be able to be used. Such a configuration of the output module 502c, in combination with the calibration output module 502b may be used to ensure the device continually operates at desired performance levels.

Turning to FIG. 5D, output module 502d is similar to output module 502a and provides optical devices 522. The optical devices 522 may be similar to the optical devices 514 of output module 502a, however they may provide a larger range of operation or adjustability. For example, the optical devices 522 may provide a greater depth of focus compared to the optical devices 514 of output module 502a.

Turning to FIG. 5E, output module 502e is similar to output module 502a however, the output module includes additional processing resources, depicted as a graphical processing unit (GPU) 524. The additional processing resources may be used by the base device, for example to process image data captured by other components. The electrical interface connection between the input module 502e and the base device may provide a high-speed and high-bandwidth communication interface.

Turning to FIG. 5F output module 502f is similar to output module 502a, however the module 502f includes an alignment device 526 that may provide one or more alignment features, depicted as three black diamonds, that can be targeted by imaging functionality of the device. The alignment features of the alignment device 526 may be located at precisely known locations on the alignment device. By imaging the alignment features, other imaging components of the device may be aligned with the known position of the alignment features. The alignment device 526 is depicted as being arranged between optical port 504 and the optical device(s) 514, however it could also be arranged between the optical device(s) 514 and optical port 508, allowing the optical devices 514 to help in focusing imaging devices on the alignment features. The alignment features may be arranged in a location that can be imaged by imaging devices without impacting imaging of the patient's eye.

Turning to FIG. 5G, output module 502g is similar to output module 502a but comprise an input optical port 528 and associated input physical interface connection 530 and electrical interface connection 532. The optical port 528 is optically coupled to an optical path connecting it to a beam combiner/splitter 534 that combines the input path with the optical path from the base device. The output module 502g may allow additional input modules to be coupled to the output module 502g. An input module may include an illuminating light source and video camera sensor that may be used to provide a video of the patient's eye during examination, and possibly treatment.

Turning to FIG. 5H, output module is 502h is similar to output module 502a but further includes a physical connector 536 that may be used to connect to other devices. For example, during examination and/or treatment a patient may place a special lens on their eye with one end of the lens providing a feature for connecting with the physical connector 536. The physical connector, and special lens, may improve the delivery of therapeutic laser pulses to the patient's eye. Additionally or alternatively, the physical connector 536 may be used to connect the module to a fiber that may be used as for example a remote imaging device or an endoscope for imaging an internal portion of the patient. Additionally or alternatively, the physical connector 536 may connect to a fixture plate or structure for holding, supporting, or providing a reference point for placing, a target relative to the imaging system. The target on the fixture plate or surface may be for example an external portion of a patient such as a head, arm, hand, chest, back leg, or foot.

Turning to FIG. 5I, an output module 502i provides a display 538. The display may be used for various functionality such a displaying information to a patient, displaying media to a patient for entertainment, including videos, movies, tv/shows, video games, etc. Although not depicted, the output module may be coupled to a remote control device that allows the patient to control the display. Additionally or alternatively, the display may be used as part of the imaging/treatment processes for example by providing a target for the user to focus on or identify for example for a periphery test, text to read, etc.

Turning to FIG. 5J, an output module 502j is depicted, which is similar to module 502a. The output module 502j provides scanning optics that can be used to scan light across a target or otherwise adjust a location that the light is focused at. The scanning optics may provide scanning along one or both the X and Y axis. The scanning optics may include for example one or more spinning mirrors, micro electro-mechanical (MEM) mirror devices, galvanometers, resonant scanners etc.

Turning to FIG. 5K, output module 502k is similar to output module 502i. Rather than providing a display, the module 502k provides a fixation target 542 which may be an LED light along with one or more optical elements for changing an apparent location of the light source allowing the location of the patient's gaze to be adjusted.

Turning to FIG. 5L, output module 502I is similar to output module 502j. Rather than providing scanning optics, module 502I provides dispersion compensation optics 544. The dispersion compensation optics may be controllable in order to compensate for varying dispersion that may occur within a target, such as a patient's eye.

Turning to FIG. 5m output module 502m is similar to output module 502j. Rather than providing scanning optics, module 502m provides a tunable lens 546. The tunable lens may be controllable in order to for example adjust a depth of focus of light passing through the tunable lens.

Turning to FIG. 5n output module 502n is similar to output module 502j. Rather than providing scanning optics, module 502n provides adaptive optics 546. The adaptive optics may be for example a spatial light modulator and may controllable in order to for example compensate for distortions in the optical system.

Turning to FIG. 5O, the input module 502o does not include optical components, but instead is used as a physical adapter. In addition to the output connection comprising the optical interface 504, physical interface 506 and electrical interface 510, the output module provides another connection comprising a physical interface 550, the output optical interface 508 and an electrical interface 552. The optical interfaces 504, 508 are optically coupled together. The electrical interfaces 510, 552 may be connected together. Although not depicted, the adapter may also include a controller. The controller may convert the signals between the electrical interfaces if necessary as well as provide information about the module to other connected modules or devices.

The above output modules have been described individually. It is possible for one or more components described with reference to one output module to be combined with components from one or more other output module. Similarly, components described with regard to a single output module may be provided in separate modules. Further, although the above has described input modules and output modules separately, it is possible for components described with respect to an input module could be used as components of an output module and for components described with respect to an output module to be used as components of an input module.

The above has described various examples of different input modules and output modules. Although described separately, components of different input modules may be combined together in a single input module. Similarly, components of different output modules may be combined together into a single output module. One or more of the input modules and output modules may be used with a base device to provide various ophthalmology functionality, such as imaging of a patient's pupil, a virtual ophthalmoscope providing 2D imaging of the fundus of the patient's eye, SLO imaging functionality, OCT imaging functionality, Laser treatment functionality, possibly using one or more different lasers such as femtosecond lasers, continuous wave lasers, etc. The different arrangements of the base device, input modules, output modules and software components used in operating the components may be combined together in various ways to provide various functionality. The input modules, output modules and software components may be provided as different kits that can be used to provide diagnosis functionality of one or more diseases or conditions as well as treatment of diseases or conditions. The table below provides an example of how different input modules, output modules and software components may be combined with a common base device to provide different functionality. Different input modules, output modules as well as operating software, may be combined together into kits for different applications. The input and output modules can provide new physical abilities to the base device such as providing a laser or different type of laser as well as particular optics allowing focusing on different parts of the eye. While the input modules and output modules can provide different physical capabilities, different software components may provide different software functionality. For example, the base software may allow for the manual evaluation of images and planning of treatment plans, while a different software component may provide machine learning (ML) components for providing automated evaluation and treatment planning. In the trailing table it is assumed that the base device provides SLO imaging functionality; however, it is possible that the base device could provide different functionality.

TABLE 1

| | Input | Output | Software |
|---|---|---|---|
| Application | module(s) | module(s) | component(s) |
| Central | Treatment laser 1 | Base model | Base mode/ML |
| photocoegulation | | | Auto |
| Peripheral | Treatment laser 1 | Module 1 | Base model/ML |
| Photocoegulation | and Lens Kit 1 | | Auto |
| Vitreous with | Treatment laser 2 | Module 2 | Base model/ML |
| femtosecond | and Lens kit 2 | | Auto |
| laser/YAG laser | | | Floater- Auto |
| Glaucoma with | Treatment laser 3 | Module 3 | Base model/ML- |
| femtosecond laser | and Lens kit 3 | | Auto |

table showing different arrangements of component kits providing different functionality

The above has described various medical imaging devices and systems. The system has been described as including a base device along with one or more connected input module and output modules that can operate with the base device. One or more kits may be provided that incorporate one or more input modules, output modules as well as an authorization to use the module and associated software functionality in the kit in order to allow the module to be enabled with the base device. In addition to a module and authorization to enable the module a, a kit may be provided with a base device. The kits may be associated with one or more applications.

FIG. 6 depicts components of a further ophthalmology system. The system 600 is depicted as including an ophthalmology base device 602 along with a connected input module 604 and output module 606. The combined base device and input/output modules may provide diagnosis and treatment functionality for diagnosing and treating one or more ocular conditions. The ophthalmology device 602 may be coupled to local a user interface device, depicted as a display 608, that allows a user such as an ophthalmologist, or other trained professional to interact with the device. Although depicted as a display, the interface may be provided in various ways including using separate computer device, a mobile computing device such as a mobile phone or tablet or possibly immersive display technology such as a virtual reality headset. The controller of the base device 602 may comprise a central processing unit (CPU) 610 or other type of controller such as an FPGA or ASIC, along with memory 612, possibly non-volatile storage 614 and one or more input/output connections for coupling one or more devices, such as display 608, to the processor. The input/output devices may include one or more wireless radios such as Wi-Fi® and/or Bluetooth® radios, wired Ethernet adapters, graphical displays and various input components including touch screens, keyboards, mice, vocal interfaces, gesture based interfaces, etc. The memory 612 may store instructions which when executed by the processor configure the base device to provide various functionality including for example, module enrollment and verification functionality 618, calibration functionality 620, ophthalmology functionality 622, remote operation functionality 624, and patient management functionality 626.

The base device 602 may operate in a standalone environment or may be connected to other devices through one or more communication networks 628. The other devices may include, for example, computing devices 638 in remote locations that provide remote operation functionality 632. Although depicted as a computer, remote computing devices may include a variety of computing devices, including, for example a mobile phone or tablet or possibly immersive display technology such as a virtual reality headset. The base device 602 may be in communication, via the network 628, with one or more back end servers 634 or computing devices that may provide module enrollment and verification functionality 636 along with a data structure storing module data 638, a data structure storing patient data, as well patient management functionality 640.

The module enrollment and verification functionality 618 may provide functionality that enrolls attached modules for use with the base device, and possibly with the remove server 634. The enrollment process may be initiated when a base device detects a new module is connected and/or when the base device is started or re-started, or optionally on demand from a user. The enrollment process may include the base device determining unique identifiers of the attached modules for example using an RFID reader, communication path with the attached module, or other ways. The enrollment process may use the unique identifier to determine information about the module, or the module information may be provided along with the unique identifier. The module information may be provided by the attached module or possibly retrieved by the base device. For example, the base device may communicate the unique ID of the connected modules to a remote server 634, which returns the module information stored in association with the unique ID to the base device. The module information may provide information that can be used to determine the compatibility of modules as well as verify that the device can provide certain functionality using the attached modules. For example, the enrollment process may retrieve module information about a connected input module and output module and may determine that the input and output modules are compatible with each other and verifies that the input and output modules, as well as the software on the base device can provide certain functionality such as detection and treatment of a certain disease. The module information for each module may include information about the module such as a broad classification of the module, operating parameters of the module, other required modules or components required to operate correctly, as well as other limitations such as incompatible devices or components, and possibly expiry information of the module.

The use of authentic modules manufactured by known, and identifiable, manufacturers may be desirable for the ophthalmology device. It is possible to use cryptographic processes to ensure that the base device along with any connected input/output modules and software components are authentic. For example, the input module may be associated with a public and private encryption key that can be used to verify that the module is authentic. Similarly, the input and output modules may use a similar cryptographic process to verify that the base device the modules are connected to is an authentic base device. If the modules are verified to be authentic, they may be prevented from operating, or may be caused to operate in a different mode, which could for example operate only basic components that could not pose a safety risk.

Once the connected modules have been registered with the base device, and possibly the remote server, the modules may be calibrated using calibration functionality 620. The calibration process provided by the calibration functionality 620 may depend upon the attached modules and the base device and possibly an operation mode of the device. The calibration process can configure the optical components of the attached components in order to operate within the operational parameters for the particular operation mode. For example, if the device is operating in a diagnosis mode, the calibration process may perform a coarse alignment of the imaging components but may not align the therapeutic laser with the imaging components as it is not used in the diagnosis mode. In addition to aligning one or more components, the calibration functionality may also calibrate other operating parameters of the devices such as a frame rate, bit depth, and resolution of captured images for one or more of connected imaging devices. Other operating parameters may include optical parameters such as power levels, frequency spectrums, pulse durations, etc.

Once the device and attached modules are calibrated, the device may operate in one or more operational modes verified to work with the attached components. The operational modes may provide various ophthalmology functionality. For example, the ophthalmology functionality 622 operate the attached modules to provide diagnosis functionality in diagnosing one or more ocular conditions such as age-related macular degeneration (AMD), wet AMD, diabetic retinopathy, glaucoma, floaters, vitreomacular traction, among other diseases. The functionality may allow the disease condition to be automatically identified within captured image data. A professional may evaluate the identified disease condition within the image data, and may possibly specify a treatment plan that can specify treatment locations and treatment parameters for treating the condition. The treatment plan may be stored in association with patient data. Alternatively, the device may provide functionality for automatically generating a treatment plan for the patient that can be subsequently verified by a professional.

The device may be able to operate in a remote operation mode that allows a professional to operate the device from a remote location. The remote location may be a physically close location, such as in an adjacent room, or may be a physically distant location such as in another city or country. Remote operation functionality 624 on the device 602 cooperates with the remote operation functionality 632 on the remote computing device 630. The remote operation functionalities may communicate remote commands as well as provide a remote graphical interface providing feedback to the user on the remote device. The remote operation functionalities 624, 632 may include functionality for periodically or continually testing the quality of the communication channel between the device 602 and remote device 630. The quality of the communication channel may be characterized by an upload bandwidth, download bandwidth, round trip time or ‘ping’, and jitter. Depending upon the operation mode of the device, the operation of the device may be suspended, or changed. For example, if the device is operating in a treatment mode and the quality of the communication channel drops below a required quality threshold, the treatment may be suspended and the device operation mode switch to one in which treatment is suspended but imaging for diagnosing and treatment planning may still be performed. Similarly, if the device is operating in an imaging mode, and the communication channel quality decreases, the remote operation functionality may change a bandwidth used by the user interface. For example, the reduced bandwidth user interface may transmit lower quality images to reduce demands on the communication channel.

In addition to the ophthalmology functionality, the device 602 may also provide patient management functionality 626 that provides an interface for users managing patients and their interactions with the device 602. The patient management functionality may provide a professional with an interface for capturing patient details such as patient name, demographics, etc. The patient management functionality may also provide scheduling functionality for scheduling patient's visits which may include accounting for an amount of time estimated for a procedure being scheduled. The patient management functionality may also provide an interface for storing and viewing images from the device, additional information related to images such as map of regions, as well as treatment plans and images of before and after treatment, etc. Although described as being provided by the base device 602, the patient management functionality could be provided by other devices such as the remote device 638, or back end server 634, or a combination of the devices.

FIG. 7 depicts an illustrative database for storing module data. The module data depicted in FIG. 7 is only illustrative and additional information may be stored and the information may be stored in different structures from those described. The database 702 may be for example the module data storage structure 638 described above. The database 702 stores a plurality of different tables that each store a plurality of records. The tables are arranged as an identification table 704 that stores information identifying individual components of the system, whether the base device, input modules, output modules, or software components. The identification table 704 may store records that include information on the component such as a unique ID or serial number of the component, a maker of the component, and a model of the component. The table may also store encryption information associated with the component such as a public key associated with the component. Depending upon the cryptographic techniques used, the encryption information may be stored as one or more one-time encryption keys or other information used for cryptographically securing communication and verifying the identity of the module.

The database 702 may further store compatibility information in a compatibility table 706. The compatibility table may store the compatibility using the make and model as a key to link the information with records in the identity table 704. The compatibility information may provide information on both the hardware compatibility and software compatibility of the component. The compatibility information may be specified in various ways but is depicted as specifying the requirements for the component to work correctly as well as limitations of the components. As an example, an output component may be designed to provide focusing optics for a treatment laser and may work with a wide range of treatment lasers. The requirements may indicate the different lasers that can function with it. Additionally the limitations may specify limitations of parameters for the component. For example while the illustrative output module may work with a wide range of different lasers the optics may only function within a specific frequency band of the light spectrum and may have a maximum power rating. These limitations may be applied to the input modules that are used with the device. Similar compatibility information may be stored for software components.

The database may also store calibration data or other data associated with the individual components in a details table 708. The records in the details table may be linked to records in the identity table 704 using the unique identifier or serial number. The details information may include information such as the date of manufacture as well as calibration information. For the optical devices that may be used in the treatment of patients, it is desirable to ensure that the components are operating within the specified parameters and as such the database may store information on when the component was calibrated, as well as re-calibrated. For example a treatment laser may be calibrated and certified when manufactured and the component may require re-calibration and certification after a certain amount of time, or a certain amount of operations. The details information may include additional information such as purchasing information, owner information, warranty information, etc.

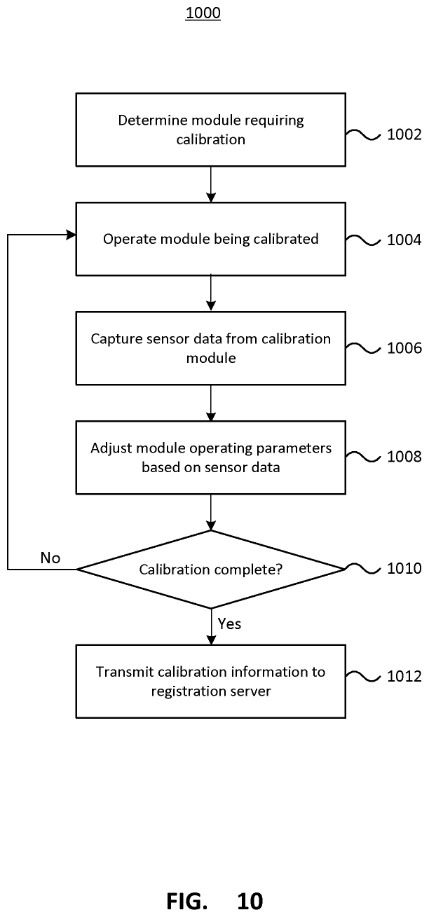

FIG. 8 depicts a method for using an ophthalmology system. The method 800 may be performed by the base device at various times, such as during startup, prior to use with a new patient, or as desired by an operator. The method 800 enables the attached modules (802) which may be achieved in various ways. For example the base device may query attached modules over the electrical connections, or other communication paths in order to retrieve module identification from each of the attached modules. Enabling the modules may also include a process for verifying that the module is certified or approved for operation with the base device. Enabling the modules may be done on all attached modules or the attached modules may be identified and presented to the operator in order to identify which components should be enabled. Such a process may be beneficial in cases in which use of a component requires payment based on the amount of times it is used, etc. Additionally or alternatively, modules may be enabled or disable in dependence upon an expiry of the module. A module may expire, for example after a certain length of time or an amount of use. The expiry may be used to enable/disable a module. An expired module may be thrown out, recalibrated, recertified, repaired, replaced, etc.

Once the modules are enabled, an operator may select an operation mode based on the enabled models. For example, the operation modes may include, modes for evaluation, screening, diagnosis and treatment. Further, the operation modes that may be provided may be dependent upon the type of disease or condition being evaluated, screened, diagnosed or treated. The device may be configured with components that can treat one or more of AMD, VMT, diabetic retinopathy, Glaucoma, floaters, retinal tears. The device may also be configured with kits of different module and/or components such as software components to provide additional functionality such as pupil imaging, a virtual ophthalmoscope, etc.

Once the operation mode is selected, for example treating a specific disease, the compatibility of the enabled components is validated for use together in the specific operation mode. This validation may include verify that the operating parameters of all components are within threshold limits for the operating mode and that the required software components are available for the device. If the operation mode is not valid (No at 808), for example one or more of the components cannot operate together, the method may either notify the operator or return to select a different mode of operation (804) or possibly enable other components (802). If the selected operation mode is valid, that is the enabled modules/components can operate to provide the selected operation mode, patient data may be retrieved (810) for the current patient. The patient data may include for example previously captured images of the patient, as well as possible treatment plans for the patient, which may be specified as a map of regions for treatment. The retrieved patient data, such as a previously captured image of the patient's eye may be compared to a currently captured image of the patient's in order to attempt to register the images together (812). The image registration process may be done using for example vein segmentation on the images. The registration may ensure that the correct patient information is being used for the current patient. Alternatively, an image of the patient's eye may be captured and used as a biometric identifier to retrieve the associated patient information. Regardless, the registration process ensures that the correct patient information is retrieved for the current patient. If the patient is being treated for a diseases or condition, the registration may also be done on the treatment plan. Once the image registration is completed, the operation mode of the device may be enabled (814) and the evaluation, screening, diagnosing and/or treating performed.

The image registration may provide a metric used to evaluate a quality of the registration between the two images, which may be captured using the same or different modalities as well as being captured at the same or different times. Validating the image registration may identify sets of landmarks, which may be coordinates, within the images being registered. The landmarks may be identified using ORB (Oriented Fast and Rotated BRIEF) processing, SIFT (Scale-invariant feature transform) processing and/or HOG (histogram of oriented gradients) processing. The identified landmarks are associated with feature vectors, with each landmark comprising a vector of N features. The landmarks and associated features of the images are compared in order to match landmarks across the images. The average and/or maximum distance between the closest matched landmarks, which may be determined as the Euclidean distance between the feature vectors of the landmarks being compared, may be used as a metric for accuracy of the registration between the two images. Additionally or alternatively, the number of landmarks match within a threshold feature distance, vs unmatched features may be used a metric for the quality of the registration. If the registration between images is of sufficient quality, a mapping that transforms matched landmark positions in one of the registered images to the positions of the corresponding matched landmarks in the other images may be determined. The mapping may include one or more linear or non-linear transformations.

FIG. 9 depicts a further method for using an ophthalmology system. The method 900 is similar to method 800 described above. The method 900 begins with the base device collecting configuration information of modules (902). The configuration information may include details about the base device, connected modules, as well as software components on the device. The configuration information is communicated to a registration server (904) and receives a validation message for modules (906) from the registration server. The registration server may be a remote server or may be a local server or computing device. Alternatively, the functionality described as being provided by the registration server may be provided within the system itself such as by the controller of the base device. The validation message may indicate which modules are validated for operation, for example possibly as a result of having a subscription to use the modules, or the modules being within a certified operational window. Additionally, the validation message may include information on possible operational modes that are validated to work with the components. The possible operational modes may also specify operating parameters of one or more of the modules/components in order to use the particular operating mode. Once the validation message is received, possible operation modes for the valid modules may be determined (908). If the validation message includes details about possible operation modes, the determination may be done by retrieving the information from the message. Alternatively, if the validation message does not include possible operation modes, the device may determine which operation modes are possible using the modules/components determined to be valid from the validation message. An operation mode is selected from the possible operation modes (910) and the modules calibrated for the selected operation mode (912). The calibration may include configuring one or more operating parameters of the modules/components as well as co-aligning one or more of the modules or components as may be required by the operating mode. Once the components are calibrated, the operation mode may be enabled (914). Prior to enabling the operation mode, the device may register previously captured patient images with currently captured patient images to ensure the correct patient data is being used with the patient as described above with reference to FIG. 8.

When the base device communicates the configuration information to the registration server, the configuration information is received (916) and for each module (918), or component, in the configuration information, the module data is validated (920) to validate that the module/component may be used. The next module (922) is validated until all of the modules have been processed. The compatibility information is retrieved for the valid modules/components (924) and the compatibility information used to determine possible operation modes for the modules (926). Once the modules are validated and the possible operation modes determined, a validation message may be constructed and returned to the base device (928). The validation message may indicate which modules/components were validated and may also include the possible operation modes that may be used with the valid modules.

FIG. 10 depicts a method for calibrating an ophthalmology system. The method 1000 may use a calibration module such as calibration module 502b described above with reference to FIG. 5B. The method 1000 may be used to calibrate one or more modules. The method 1000 determines which module requires calibration (1002). The determination of which modules need calibration may be determined based on which modules may be used for an operation mode, if modules have been newly installed, if the modules need to be re-calibrated or certified after a length of time or amount of use. Once the module(s) requiring calibration are determined, the module(s) being calibrated are operated (1004) and sensor data from calibration components, which may be provided in a calibration module or in the device or other modules, are captured (1006). The operating parameters of the module being calibrated, as well as possible other components or modules, may be adjusted based on the sensor data (1008). For example, the sensor data may be used to adjust a tunable lens in order to calibrate the alignment and/or focus of an imaging device or treatment laser. Additional calibration may include, for example adjusting power levels of devices, adjusting frequency spectrums, and adjusting other optical devices. If the calibration is complete (No at 1010), the method may return to operate the module (1004) capture sensor data (1006) and further adjust operating parameters (1008). If the calibration is complete (1010), the calibration information may be transmitted to a registration server (1012) for storing details about the calibration, which may be used to ensure the modules are being operated within appropriate operating parameters.

It will be appreciated by one of ordinary skill in the art that the system and components shown in FIGS. 1-10 may include components and/or steps not shown in the drawings. For simplicity and clarity of the illustration, elements in the figures are not necessarily to scale, are only schematic and are non-limiting of the elements structures. It will be apparent to persons skilled in the art that a number of variations and modifications can be made without departing from the scope of the invention as defined in the claims.

Although certain components and steps have been described, it is contemplated that individually described components, as well as steps, may be combined together into fewer components or steps or the steps may be performed sequentially, non-sequentially or concurrently. Further, although described above as occurring in a particular order, one of ordinary skill in the art having regard to the current teachings will appreciate that the particular order of certain steps relative to other steps may be changed. Similarly, individual components or steps may be provided by a plurality of components or steps. One of ordinary skill in the art having regard to the current teachings will appreciate that the components and processes described herein may be provided by various combinations of software, firmware and/or hardware, other than the specific implementations described herein as illustrative examples.

The techniques of various embodiments may be implemented using software, hardware and/or a combination of software and hardware. Various embodiments are directed to apparatus, e.g. a node which may be used in a communications system or data storage system. Various embodiments are also directed to non-transitory machine, e.g., computer, readable medium, e.g., ROM, RAM, CDs, hard discs, etc., which include machine readable instructions for controlling a machine, e.g., processor to implement one, more or all of the steps of the described method or methods.

Some embodiments are directed to a computer program product comprising a computer-readable medium comprising code for causing a computer, or multiple computers, to implement various functions, steps, acts and/or operations, e.g. one or more or all of the steps described above. Depending on the embodiment, the computer program product can, and sometimes does, include different code for each step to be performed. Thus, the computer program product may, and sometimes does, include code for each individual step of a method, e.g., a method of operating a communications device, e.g., a wireless terminal or node. The code may be in the form of machine, e.g., computer, executable instructions stored on a computer-readable medium such as a RAM (Random Access Memory), ROM (Read Only Memory) or other type of storage device. In addition to being directed to a computer program product, some embodiments are directed to a processor configured to implement one or more of the various functions, steps, acts and/or operations of one or more methods described above. Accordingly, some embodiments are directed to a processor, e.g., CPU, configured to implement some or all of the steps of the method(s) described herein. The processor may be for use in, e.g., a communications device or other device described in the present application.

Numerous additional variations on the methods and apparatus of the various embodiments described above will be apparent to those skilled in the art in view of the above description. Such variations are to be considered within the scope of the teachings of the disclosure.

1. An ophthalmology device comprising:
a first optical device comprising a first light source and first imaging sensor for capable of generating image data using the first light source;
a first input coupling comprising:
a physical interface connection for aligning and securing an input optical module; and
an optical interface connection;
a first optical output;
an optical pathway coupling the first optical device and the optical interface connection of the first input coupling to the first optical output; and
an output coupling providing a physical interface connection for aligning and securing a third optical device.

2. The ophthalmology device of claim 1, further comprising a controller for controlling operation of the ophthalmology device and providing one or more of:
calibration functionality for enrolling connected input modules and output modules for use with the ophthalmology device;
image registration functionality; and
retina tracking functionality.

3. The ophthalmology device of claim 2, wherein the first input coupling further comprises an electrical interface connection.

4. The ophthalmology device of claim 3, wherein the electrical interface connection provides a communication channel to the controller of the ophthalmology device.

5. The ophthalmology device of claim 1, further comprising:
a first tunable lens for the first optical device within the optical pathway; and
a second tunable lens for the first input coupling within the optical pathway.

6. The ophthalmology device of claim 5, wherein the first tunable lens and second tunable lens are controlled by a controller in order to co-align light from the first optical device and the optical interface connection of the first input coupling.

7. The ophthalmology device of claim 1, further comprising a second input coupling comprising:
a physical interface connection for aligning and securing a fourth optical device; and
an optical interface connection.

8. The ophthalmology device of claim 7, wherein the second input coupling further comprising an electrical interface connection.

9. The ophthalmology device of claim 1, wherein the output coupling further comprises an electrical interface connection.

10. The ophthalmology device of claim 1, wherein the optical pathway comprises one or more of:
an optical splitter/combiner;
an optical circulator;
a filter;
a galvanometer;
a tunable lens;
a lens; and
a grating.

11. An ophthalmology system comprising:
a first optical device comprising a first light source and first imaging sensor capable of generating image data using the first light source;
a first input coupling comprising:
a physical interface connection for aligning and securing an input optical module; and
an optical interface connection;
a first optical output;
an optical pathway coupling the first optical device and the optical interface connection of the first input coupling to the first optical output;
an output coupling providing a physical interface connection for aligning and securing a third optical device; and
an input optical module secured to the first input coupling.

12. The ophthalmology system of claim 11, wherein the input optical module comprises at least one of:
an imaging device including a light source and sensor, optically coupled to the optical interface connection of the first input coupling; and
a therapeutic laser.

13. The ophthalmology system of claim 12, wherein the input optical module further comprises one or more of:
an optical device;
a pilot laser;
a graphical processing unit; and
an additional input coupling.

14. The ophthalmology system of claim 13, wherein the input optical module provides one or more of:
a 2D imaging system;
a scanning laser ophthalmoscopy (SLO) imaging system;
an optical coherence tomography (OCT) imaging system; or
a therapeutic laser treatment system.

15. The ophthalmology system of claim 11, further comprising an additional input optical module.

16. The ophthalmology system of claim 11, further comprising an output optical module.

17. The ophthalmology system of claim 16, wherein the output optical module comprises one or more of:
an optical device;
a graphical processing unit;
an additional input coupling;
an additional output coupling; or
a sensor.

18. A kit for an ophthalmology device comprising:
a first optical module comprising:
one or more optical components for providing ophthalmology functionality to the ophthalmology device;
a physical interface connection for mechanically securing the first optical module to the ophthalmology device;
an optical interface connection for optically coupling at least a portion of the one or more optical components to the ophthalmology device; and
a module identifier for uniquely identifying the first optical module; and
an authorization for use of one or more software components by the ophthalmology device to provide the ophthalmology functionality using the first optical module identified by the module identifier.

19. The kit of claim 18, wherein the first optical module comprises one of an input module and an output module.

20. (canceled)

21. The kit of claim 18, wherein the kit comprises at least one input module and output module suitable for performing one or more of:
central photocoagulation;
peripheral photocoagulation;
vitreous treatment with femtosecond laser;
vitreous treatment with YAG laser; or
glaucoma treatment with femtosecond laser.

22.-30. (canceled)
